(19) United States
(12) Patent Application Publication
(10) Pub. No.: US 2026/0182848 A1
(43) Pub. Date: Jul. 2, 2026

(54) Transjugular Intrahepatic Portosystemic Shunt Pressure Measurement Systems and Methods

(71) Applicant: Baylis Medical Technologies Inc., Mississauga (CA)

(72) Inventors: Amanda Centazzo-Colella, Bowmanville (CA); Patrick Ryan, Toronto (CA); Michael Dallimore, Barrie (CA); Amanda Hartley, Caledon (CA)

(21) Appl. No.: 19/129,884

(22) PCT Filed: Nov. 14, 2023
(86) PCT No.: PCT/IB2023/061492

§ 371 Date: May 14, 2025

Related U.S. Application Data
(60) Provisional application No. 63/426,116, filed on Nov. 17, 2022.

(51) Int. Cl. A61B 5/0215; A61B 5/00

(52) U.S. Cl. CPC A61B 5/02152; A61B 5/6851; A61B 5/6853; A61B 5/6869; A61B 5/6876; A61B 5/7271; A61B 5/7405; A61B 5/742; A61B 5/746; A61B 2505/05

(57) ABSTRACT

An apparatus and method of using the apparatus are disclosed for measuring a pressure differential during a transvascular procedure such as a Transjugluarl Intrahepatic Portosystem Shunt (TIPS) procedure, or a Direct Portosystemic Intrahepatic Shunt (DIPS) procedure. The present invention provides a telescoped assembly comprising a first pressure measurement mechanism and a second pressure measurement mechanism to measure a pressure differential between a first pressure located in a first blood vessel and a second pressure located in a second blood vessel, while substantially maintaining the position of the first and second pressure measurement mechanisms, thereby reducing the risk of damage to the liver and surrounding tissues.

### TECHNICAL FIELD

The disclosure relates to systems and methods to measure pressure in the human body, and more particularly to systems and methods for measuring a portosystemic gradient during a Transjugular Intrahepatic Portosystemic Shunt (TIPS) procedure.

### BACKGROUND

The portosystemic gradient (PSG), i.e. the difference between the pressure in the portal and venous systems, is a critical parameter that must be determined in order to complete a TIPS procedure. The PSG is found by measuring the venous pressure and portal pressure and calculating the difference between the two values. The venous pressure (also referred to as venous, systemic, or hepatic pressure) is measured in the hepatic vein, the inferior vena cava (IVC), or the right atrium. The portal pressure is measured in the portal vein once the portal vein has been accessed.

During a TIPS procedure, a baseline PSG value is measured before a tract is created from the hepatic vein, through the liver, to the portal vein. How and when this measurement is taken depends on the procedural approach. Measurements in the systemic venous system are relatively easy as the system is readily accessible with transjugular access. However, portal measurements are more difficult as the portal vein is not connected directly to the heart or venous system and an indirect measurement approach is needed. Commonly, after a needle has punctured through the liver from the hepatic to the portal vein, a small catheter is passed over the needle and delivered to the portal vein, the needle is removed, and the now empty lumen of the catheter is used to measure the portal pressure. In some procedures, the portal vein may be accessed directly via the abdomen and in this way the pressure gradient can be measured before puncture of the liver. Another method involves measuring the portal pressure indirectly prior to puncture by using a balloon to occlude the hepatic vein. Various studies have demonstrated a correlation between the occluded pressure of the hepatic vein and the portal vein pressure.

Following creation of a tract between the hepatic vein and portal vein, a stent is placed between the two vessels. After the stent has been delivered, an initial expansion of the stent using a balloon forms the intrahepatic TIPS tract. At this point a second (post-tract creation) PSG is measured. If the difference between the hepatic venous pressure and portal pressure has fallen below a predetermined threshold (typically <12 mmHg) this is considered the endpoint of the procedure. If the PSG remains high, the balloon is re-delivered, and the stent is expanded to a larger diameter. The balloon is removed, and the PSG is then measured again.

### BRIEF DESCRIPTION OF THE DRAWINGS

In order that the invention may be readily understood, embodiments of the invention are illustrated by way of examples in the accompanying drawings, in which:

FIG. 1A is an illustration of the anatomy of a liver and surrounding blood vessels;

FIG. 1B is an illustration of an elongate medical device spanning a tract from the hepatic vein, through the liver, to the portal vein during a TIPS procedure;

FIG. 1C is an illustration of the liver showing a stent extending between the portal vein and inferior vena cava during a direct intrahepatic shunt (DIPS) procedure;

FIG. 2 is an illustration of a telescoping medical device assembly for performing at least part of a TIPS procedure in accordance with one embodiment of the present invention;

FIG. 3A is an illustration of an elongate device comprising a pressure sensor in accordance with an embodiment of the present invention;

FIG. 3B is an illustration of multiple elongate devices comprising pressure sensors in accordance with an embodiment of the present invention;

FIG. 3C is an illustration of an elongate device comprising multiple pressure sensors in accordance with an alternative embodiment of the present invention;

FIG. 3D is an illustration of an elongate device comprising a balloon in accordance with a further alternative embodiment of the present invention;

FIG. 4A is a cross-section illustration of two elongate devices forming part of a telescoping assembly in accordance with an embodiment of the present invention;

FIG. 4B is a further cross-section illustration of the two elongate devices of FIG. 4A;

FIG. 5 is an illustration of the telescoping assembly connected to a pressure measurement mechanism in accordance with one embodiment of the present invention;

FIGS. 6A(i) and 6A(ii) are illustrations of the distal end of the telescoping assembly in accordance with one embodiment of the present invention;

FIGS. 6B to 6D are illustrations of two devices forming part of the telescoping assembly in accordance with alternative embodiments of the present invention;

FIGS. 7A to 7B are illustrations of devices of the telescoping assembly comprising tapered distal ends in accordance with an embodiment of the present invention;

FIGS. 8A to 8E are illustrations the devices of the telescoping assembly with one device comprising a varying-diameter portion in accordance with an embodiment of the present invention;

FIG. 9A(i) is cross-section an illustration of a device of the telescoping assembly comprising a pressure differential mechanism in accordance with an embodiment of the present invention;

FIG. 9A(ii) is a cross-section illustration of the device of FIG. 9A(i);

FIG. 9B is an illustration of a device of the telescoping assembly comprising a pressure differential mechanism in accordance with an alternative embodiment of the present invention;

FIG. 9C is an illustration of the device of FIG. 9A(i) in use during a TIPS procedure in accordance with an embodiment of the present invention;

FIG. 9D is an illustration of a device of the telescoping assembly comprising a pressure differential mechanism in accordance with a further embodiment of the present invention;

FIG. 10 is an illustration of a device of the telescoping assembly comprising a pressure differential mechanism in accordance with a further alternative embodiment of the present invention;

FIG. 11 is a flow diagram showing a method of performing part of a TIPS procedure in accordance with an embodiment of the present invention; and

FIG. 12 is a flow diagram showing a method of performing part of a TIPS procedure in accordance with an alternative embodiment of the present invention.

### DETAILED DESCRIPTION

In one broad aspect, embodiments of the present invention comprise a telescoping assembly comprising: a first elongate member; a second elongate member moveable relative to the first elongate member when said second elongate member is inserted into said first elongate member; a first pressure measurement mechanism associated with one of the first elongate member or the second elongate member; and a second pressure measurement mechanism associated with one of the first elongate member or the second elongate member.

As a feature of this aspect, the first pressure measurement mechanism comprises a first pressure sensor and the second pressure measurement mechanism comprises a second pressure sensor.

As another feature of this aspect, the first pressure measurement mechanism comprises a pressure transmitting lumen, the pressure transmitting lumen being adapted for fluid communication with a pressure transducer, and the second pressure measurement mechanism comprises a pressure sensor.

As another feature of this aspect, the first pressure measurement mechanism comprises a first pressure transmitting lumen, the first pressure transmitting lumen being adapted for fluid communication with a first pressure transducer, and the second pressure measurement mechanism comprises a second pressure transmitting lumen, the second pressure transmitting lumen being adapted for fluid communication with a second pressure transducer.

As another feature of this aspect, the first elongate member is selected from the group consisting of: a sheath; a dilator; and a catheter, and the second elongate member is selected from the group consisting of: a dilator; a catheter; and a guidewire.

As another feature of this aspect, the first pressure sensor and the second pressure sensor are both associated with the first elongate member.

As another feature of this aspect, the first pressure sensor and the second pressure sensor are both associated with the second elongate member.

As another feature of this aspect, the pressure transmitting lumen is associated with the first elongate member, and the first elongate member is selected from the group consisting of a sheath; a dilator; and a catheter, and the pressure sensor is associated with the second elongate member and the second elongate member is selected from the group consisting of a dilator; a catheter; and a guidewire.

As another feature of this aspect, the pressure transmitting lumen and the pressure sensor are both associated with the first elongate member, and the first elongate member is selected from the group consisting of: a sheath; a dilator; and a catheter.

As another feature of this aspect, the pressure transmitting lumen and the pressure sensor are both associated with the second elongate member, and the second elongate member is selected from the group consisting of a dilator; and a catheter.

As another feature of this aspect, the pressure sensor is associated with the first elongate member, and the first elongate member is selected from the group consisting of a sheath; a dilator, and the pressure transmitting lumen is associated with the second elongate member and the second elongate member is selected from the group consisting of a dilator; and a catheter.

As another feature of this aspect, the first pressure transmitting lumen is associated with the first elongate member, and the first elongate member is selected from the group consisting of a sheath and a dilator, and wherein the second pressure transmitting lumen is associated with the second elongate member, and the second elongate member is selected from the group consisting of a dilator; and a catheter.

As another feature of this aspect, the second elongate member comprises a balloon and the first pressure sensor is operatively located on one of a distal or proximal side of the balloon and the second pressure sensor is operatively located on another of the distal or proximal side of the balloon.

As another feature of this aspect, the guidewire comprises a radiofrequency wire.

As another feature of this aspect, the guidewire comprises a mechanical wire.

As another feature of this aspect, the first elongate member defines a tapered end defining one or more apertures.

As another feature of this aspect, the second elongate member is selected from the group consisting of: a dilator; and a catheter, and the second elongate member defines a tapered end defining one or more apertures.

As another feature of this aspect, the second elongate member defines a varying-diameter portion.

As another feature of this aspect, in an advanced position of the telescoping assembly, the varying-diameter portion is spaced distally from a distal end of the first elongate member, and in a retracted position of the telescoping assembly, the varying-diameter portion substantially abuts the distal end of the first elongate member.

As another feature of this aspect, a gap is defined between a distal end of the first elongate member and the varying-diameter portion in the advanced position.

In a further broad embodiment, embodiments of the present invention comprise a system for use in an intrahepatic portosystemic shunt procedure comprising: the telescoping assembly described herein; and a monitor for displaying at least one of: a first pressure associated with the first pressure measurement mechanism; a second pressure associated with the second pressure measurement mechanism; or a pressure differential between the first pressure and the second pressure.

As a feature of this aspect, the monitor is configured to generate an alert when the pressure differential has reached a predetermined threshold.

As another feature of this aspect, the alert is an audible alert.

As another feature of this aspect, the alert is visual alert.

In a further broad embodiment, embodiments of the present invention comprise a medical device comprising: an elongate member comprising an inner wall and an outer wall; a first opening defined by the outer wall; a second opening defined by the outer wall, spaced apart longitudinally from the first opening; a lumen, defined between the inner wall and the outer wall, the lumen extending between the first opening and the second opening; and a pressure differential mechanism located within the lumen for measuring a pressure differential between a first pressure at the first opening and a second pressure at the second opening.

As a feature of this aspect, the pressure differential mechanism comprises a pressure sensor.

As another feature of this aspect, the pressure differential mechanism comprises a contrast bubble.

As another feature of this aspect, the pressure differential mechanism comprises a turbine.

As another feature of this aspect, the pressure differential mechanism comprises a balloon.

As another feature of this aspect, the elongate member is selected from a group consisting of a sheath, a dilator, and a catheter.

In a further broad embodiment, embodiments of the present invention comprise a system for use in an intrahepatic portosystemic shunt procedure comprising:

the medical device described herein; and a monitor for displaying at least one of: a first pressure associated with the first pressure measurement mechanism; a second pressure associated with the second pressure measurement mechanism; or a pressure differential between the first pressure and the second pressure.

As a feature of this aspect, the monitor is configured to generate an alert when the pressure differential has reached a predetermined threshold.

As another feature of this aspect, the alert is an audible alert.

As another feature of this aspect, the alert is visual alert.

In a further broad embodiment, embodiments of the present invention comprise a method of measuring pressure during an intrahepatic portosystemic shunt procedure, the method comprising: measuring a first pressure at a first location associated with the intrahepatic portosystemic shunt procedure within a patient's body using a first pressure measurement mechanism; and measuring a second pressure at a second location associated with the intrahepatic portosystemic shunt procedure within the patient's body using a second pressure measurement mechanism; wherein the first pressure and the second pressure are measured while substantially maintaining a position of the first pressure measurement mechanism at the first location and maintaining a position of the second pressure measurement mechanism at the second location.

As a feature of this aspect, the steps of measuring the first pressure and measuring the second pressure are performed substantially concurrently.

As another feature of this aspect, the first pressure is a venous pressure and the second pressure is a portal pressure.

As another feature of this aspect, the first location is one selected from the group of: the hepatic vein, the right atrium, and the inferior vena cava.

As another feature of this aspect, the second location is the portal vein.

As another feature of this aspect, the method comprises a step of positioning the first pressure measurement mechanism at the first location and positioning the second pressure measurement mechanism at the second location.

As another feature of this aspect, the method further comprises a step of creating a channel through tissue in order to access the second location.

As another feature of this aspect, the channel is created using radiofrequency energy.

As another feature of this aspect, the channel is created using mechanical energy.

As another feature of this aspect, the method further comprises a step of generating an alert when a pressure differential between the first pressure and the second pressure has reached a predetermined threshold.

In a further broad embodiment, embodiments of the present invention comprise a method of measuring a pressure differential during an intrahepatic portosystemic shunt procedure, the method comprising: measuring a pressure differential between a first pressure at a first location and a second pressure at a second location, the first and second locations being associated with the intrahepatic portosystemic shunt procedure within a patient's body, using a pressure differential mechanism.

For explanatory purposes, the systems and methods disclosed herein are generally described with reference to a transjugular intrahepatic shunt (TIPS) procedure, where a tract is created from the hepatic vein to the portal vein. However, as would be apparent to one skilled in the art, the systems and method described herein are also applicable to a direct intrahepatic shunt (DIPS) procedure, and may be applied accordingly. Certain aspects of this disclosure are also applicable to other medical procedures as well beyond TIPS and DIPS.

Current methods of measuring pressure often make use of luer-lock connectable pressure transducers. These transducers require a conduit between them and the location of the pressure measurement (e.g. a lumen) with sufficient surface area to transmit the pressure accurately.

Measuring the pressure in a TIPS procedure typically requires the removal or exchange of one or more devices in order to provide a lumen for pressure measurement. For example, after the puncture is completed, telescoped items such as a puncturing device may be removed from within larger devices, e.g. a sheath or a catheter, such that their primary lumens can be used for pressure measurement. The present inventors have conceived of novel and inventive devices, assemblies, and methods for improving pressure measurements in TIPS procedures.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention discloses a method of performing part of a TIPS procedure using a telescoping assembly, for example an assembly comprising a flexible Radio Frequency (RF) device, a catheter, a dilator, and a steerable sheath. Such an assembly is disclosed in U.S. Pat. No. 11,324,548 B2, filed by Baylis Medical Company Inc. Other systems or assemblies may be used as well and the invention is not limited in this regard. After portal vein access has been confirmed using the assembly, i.e. after a pathway has been created from the hepatic vein to the portal vein, pressure measurements are taken in the hepatic vein and portal vein.

A system or assembly, as well as a method of use thereof, is described herein that is operable to measure pressure during a TIPS procedure while mitigating one or more of the risks noted herein.

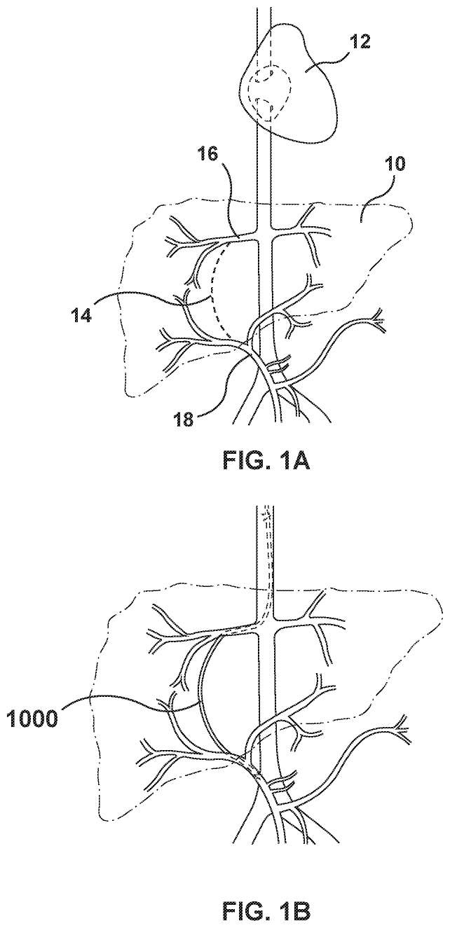

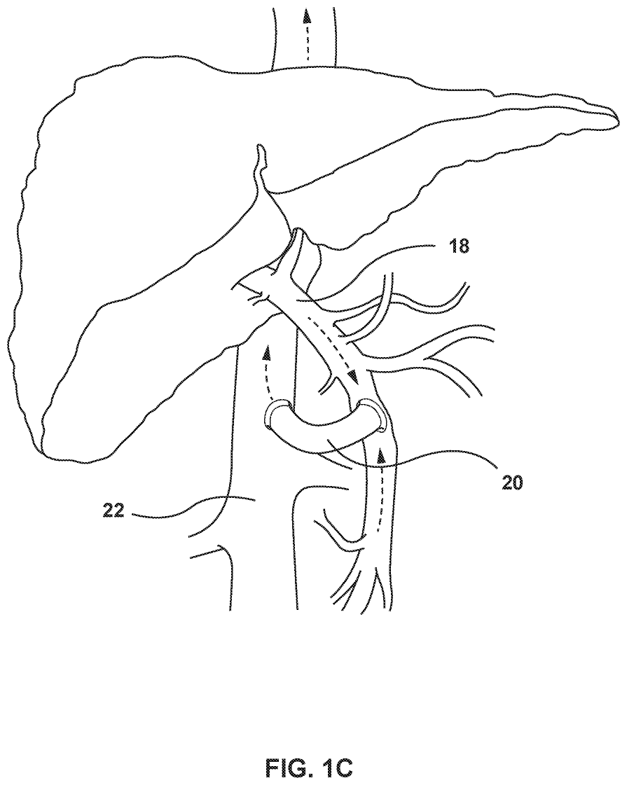

Referring to FIGS. 1A to 1C, a diagram of a liver 10 and adjoining structures is shown. FIG. 1A shows the right atrium 12, and a dashed line representing a path for a tract 14 to be created between the hepatic vein 16 and portal vein 18. FIG. 1B shows an elongated medical device assembly 1000, running from the venous system into the portal vein 18 through a tract 14 created in the liver 10, for example during a TIPS procedure. FIG. 1C shows a shunt 20 extending between the portal vein 18 and inferior vena cava 22, for example during a DIPS procedure.

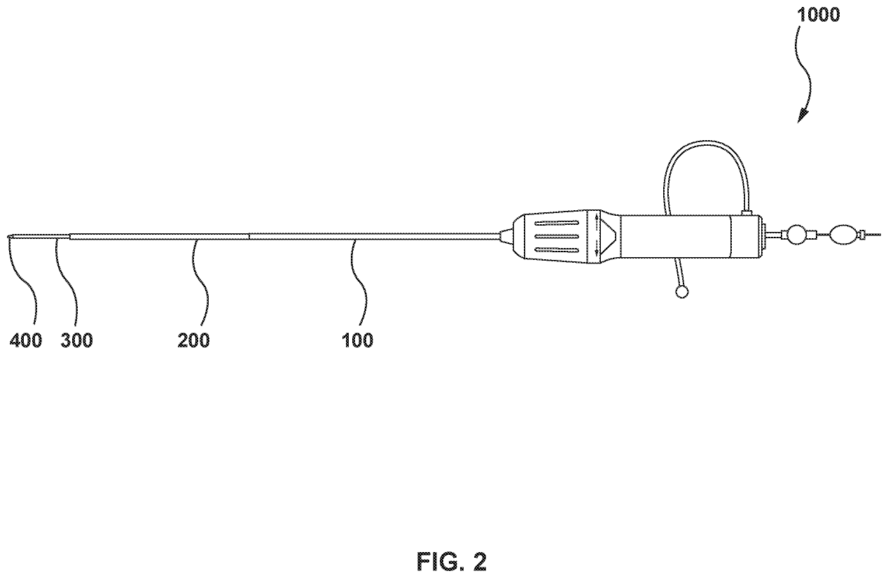

Referring to FIG. 2 in one embodiment of the present invention, a medical device assembly 1000 comprises a sheath 100 (which may also be referred to as a steerable guiding sheath), a dilator 200 (which may also be referred to as a flexible dilator), catheter 300 (which may also be referred to as a microcatheter or crossing catheter), and puncture device 400. In one embodiment, puncture device 400 is an RF wire, capable of delivering RF energy at the distal tip to cut through tissue. RF wire is connected to an energy deliver component, for example a generator, at the proximal end (not shown) (RF may also be referred to as RF guidewire). In other embodiments, puncture device 400 may be a needle, a stylet, a mechanical wire, or other guidewire that does not deliver RF energy.

In one specific example, the medical device assembly 1000 of the present invention comprises a sheath 100 such as a 10 French (Fr) steerable sheath, a dilator 200 such as a 10 Fr flexible dilator, a catheter 300 such as a 5 Fr crossing catheter, and a puncture device 400 such as a 0.035″ RF guidewire. In one such example, the telescoping assembly 1000 includes a sheath 100, a dilator 200 received within the sheath 100, a catheter 300 received within the dilator 200, and a puncture device 400 received within the catheter 300, each component being received in a telescoping arrangement relative to the others. In another embodiment, the sheath 100 may be a fixed 10 Fr sheath and the catheter 300 may be a 3 Fr-6 Fr steerable catheter. “Medical device assembly” 1000 may be referred to as “telescoping assembly” 1000.

In accordance with another embodiment of the present invention a telescoping assembly (not shown) comprises an introducer sheath, for example a 10 Fr introducer sheath, a steerable guiding sheath such as a 7 Fr steerable sheath, a flexible dilator such as a 7 Fr flexible dilator, a crossing catheter such as a 4 Fr crossing catheter, and a puncture device. In one such example, the steerable guiding sheath is received within the introducer sheath, the flexible dilator is received within the steerable guiding sheath, the crossing catheter is received within the flexible dilator, and the puncture device is received within the crossing catheter, each component being arranged in the telescoping arrangement described herein above.

For descriptive purposes, any of the sheath, dilator, catheter, or puncture device may be referred to as a “device”, for example, the same modifications and relationships between the sheath and dilator may apply between the dilator and catheter. Devices may also be referred to as either an “inner device” or an “outer device”. For example, when describing the dilator and catheter, the catheter may be positioned within the dilator, such that the catheter would be referred to as the inner device and the dilator would be referred to as the outer device.

In some embodiments, each device of the telescoping assembly 1000 is separate and fully removable from all other devices. In other words, the devices may be assembled in any suitable combination depending on the application. For example, if a procedure requires more space within one device, for example the sheath, then one or more of the other devices within the telescoping assembly, for example the dilator or the catheter, may be removed. In addition, certain devices may be “backloadable” over other devices of the assembly. For example, a dilator may be advanced proximally or removed distally over top of a catheter. For example, a dilator may be removed from the assembly without first having to remove a catheter.

Such embodiments that allow for removal of one or more components of the assembly during the course of a procedure may provide particular advantages. For example, a non-functional device may be swapped or exchanged for a new device. As a specific example, if a sensor (described below) malfunctions on one device of the assembly, that particular device may be removed and a new device with a working sensor may be inserted.

Independently movable components within the telescoping assembly provide further advantages as well. For example, if a reading on a pressure sensor (described below) at a first location is questionable, a pressure sensor on another independent device of the assembly may be moved to the first location to confirm the pressure reading. Furthermore, a distance between sensors, or between any other components of the assembly, may be modified during the course of a procedure if the assembly devices are independently moveable. Thus, for example, pressure measurements may be taken at relatively large distances between locations in blood vessels within a patient's body.

### Pressure Sensors

As noted hereinabove, current TIPS procedures typically include measuring pressure at two separate and independent vascular locations, such as the hepatic vein and portal vein. Although the two pressures can be measured independently at different times, using a single pressure measurement device moved between the two locations, this requires moving at least one component between the locations, which is undesirable for several reasons. For example, additional movements of components may damage one or more of the component and tissue of a patient's body, they may lead to less accurate measurements, and they may also prolong the procedure.

The present inventors have conceived of devices, assemblies and methods that allow for a simultaneous, two-point measurement. The present invention is particularly advantageous because it avoids unnecessary movement or exchange of components, thereby potentially improving the accuracy of the pressure measurements. Also, moving devices through the intrahepatic track can be challenging due to the stiffness properties of the liver. Such challenges and risks of tissue damage are mitigated by the present invention.

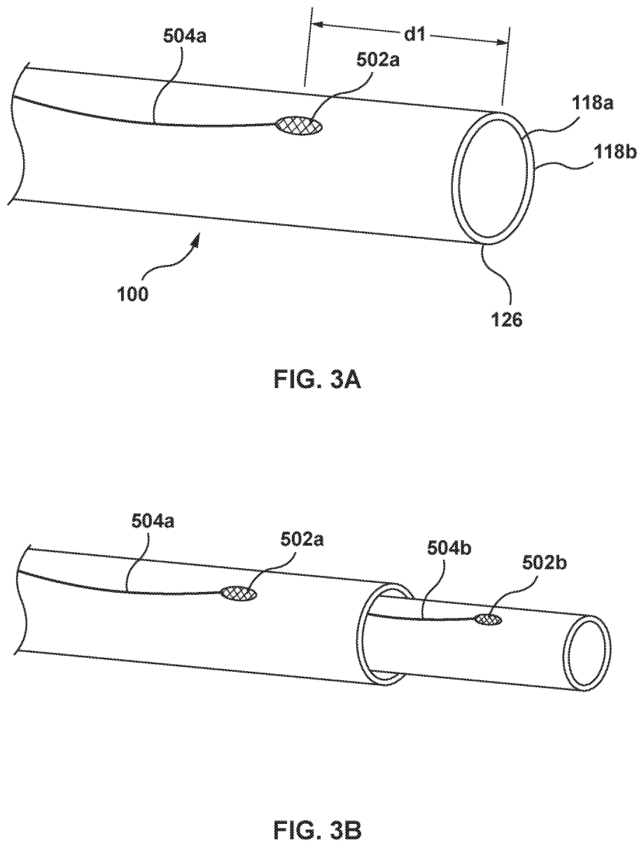

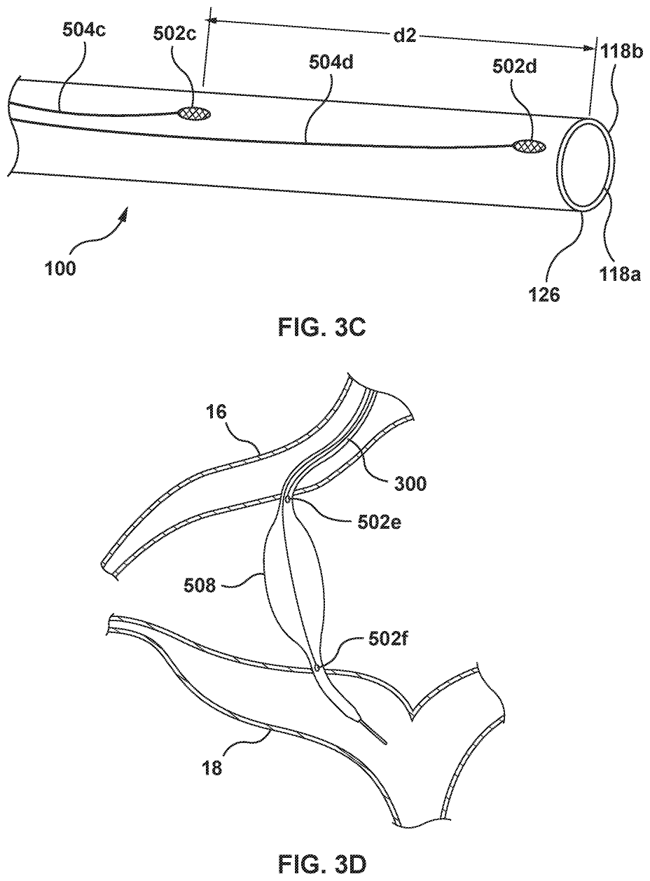

Referring to FIGS. 3A to 3D, according to one embodiment of the present invention, one or more pressure measurement mechanisms are associated with one or more devices of the telescoping assembly 1000. In one specific embodiment, a pressure measure mechanism comprises a pressure sensor 502a, located on the outer wall 118b of sheath 100.

In another embodiment, one or more pressure sensors 502 are embedded between an inner wall 118a and outer wall 118b of sheath 100. Pressure sensor 502 may be any type of sensor capable of fitting within an elongate device and capable of measuring acceptable pressure ranges, for example within 0-40 mmHG. In other embodiments any or all of dilator 200, catheter 300, or puncture device 400 may comprises one or more pressure sensors 502.

In one example of this embodiment, pressure sensor 502a is positioned on the sheath 100 in such a way that the portion of the sheath 100 that includes pressure sensor 502a can remain located in the hepatic vein throughout the TIPS procedure, for example it is positioned a certain distance D1 from the distal end 126 of the sheath. In one embodiment, D1 may be between 4 cm and 15 cm. In one specific example, D1 is between 9 cm and 11 cm.

In one embodiment, at least two devices of the telescoping assembly 1000 each comprise a pressure sensor 502, for example a pressure sensor 502a associated with sheath 100 and a pressure sensor 502b is associated with catheter 300, as shown in FIG. 3B. In such an example the pressure sensor 502b is located at or near the distal end of catheter 300. In further examples, pressure sensor 502a is located at or near to the distal end of sheath 100.

As described hereinabove, pressure sensor 502a is usable to determine a first pressure in a first blood vessel and pressure sensor 502b is usable to determine a second pressure in a second blood vessel. Specifically with respect to a TIPS procedure, the first pressure is the venous pressure, the first blood vessel in one of the hepatic vein 16, the inferior vena cava 22, or the right atrium 12, the second pressure is the portal pressure, and the second blood vessel is the portal vein 18.

In some embodiments, pressure sensors 502a and 502b allow for continuous pressure measurements throughout the TIPS procedure by maintaining the pressure sensor 502b in the portal system and pressure sensor 502a in the hepatic vein 16, inferior vena cava 22, or right atrium 12 throughout the procedure.

In other embodiments, pressure sensors 502 are embedded on other devices including the dilator 200 and puncture device 400. Pressure sensors 502 may be placed on any combination of the devices in such a manner that two pressure measurements may be taken concurrently in order to calculate the PSG, i.e., one pressure measurement in the hepatic vein 16 and one pressure measurement in the portal vein 18. As described hereinabove, multiple pressure sensors 502 allow for multiple pressure measurements at multiple locations concurrently. Additionally, in certain embodiments, multiple pressures may be measured while maintaining the position of each pressure sensor, in other words, without requiring repositioning of the devices.

Referring to FIG. 3C, in another embodiment, multiple pressure sensors 502 are associated with a single device in such a way that a first pressure measurement in a first blood vessel and a second pressure measurement in a second blood vessel may be taken concurrently using a single device. In a specific example, the first blood vessel is the hepatic vein 16 and the second blood vessel is the portal vein 18.

In one specific example, sheath 100 comprises pressure sensors 502c and 502d. Pressure sensor 502c is located a distance D2 from the distal end 126 and pressure sensor 502d is located at or near the distal end 126. In some embodiments, with specific reference to a TIPS procedure, distance D2 may be equal to or greater than the tract 14 length from the hepatic vein 16 to the portal vein 18.

In another embodiment, one of the devices, for example catheter 300′, comprises a balloon 508 and pressure sensors 502e and 502f, as shown in FIG. 3D. Pressure sensors 502e and 502f are located at or near opposing sides of balloon 508, for example on either side of the balloon 508. This allows balloon 508 to be delivered and the baseline PSG to be measured. Subsequently, as the stent is expanded using the balloon 508, a real-time uninterrupted PSG may be read. In another embodiment, balloon 508 is associated with dilator 200. In another embodiment, balloon 508 may be placed on a device separate from the telescoping assembly 1000.

In some embodiments, pressure sensors 502 are connected to one or more wire leads 504, for example 504a, 504b, 504c, and 504d shown in FIGS. 3A to 3C. Wire leads 504 run along the length of the device to the proximal end of the device, and thereafter are operable to be electrically coupled to least one monitor 506. Wire leads 504 may run along the outside of a device, or may be embedded within the device, i.e., between inner and outer walls of the device, for example in a lumen or in a sidewall. In another embodiment, wire leads 504 are positioned within a plastic jacket, which may be fused into the device or formed within the device itself. In another embodiment, pressure sensors 502 are wireless sensors that are operable to communicate with an external monitor without requiring wire leads.

Pressure sensors 502 may be connected to a dedicated monitor 506 or integrated into an RF generator (not shown). In one embodiment, monitor may display at least one or more of a first pressure, a second pressure, or a pressure differential (i.e., the PSG). In one specific example, the pressure readouts are specific to the TIPS procedure and may include specialized graphics or additional data. For example, in some such examples, monitor 506 is configured and operable to automatically alert a physician that a threshold PSG has been reached, for example through an audible or visual alert, signal, or reading. Reaching a threshold means that the PSG is below or above a predetermined pressure differential, for example below 12 mmHG. In one specific embodiment, the threshold PSG is user adjustable.

In one example, a device handle may comprise a terminal and/or electrical connection (for example a LEMO connector or similar) that plugs into a monitor 506.

### Pressure Measurement Lumens

In another embodiment, the pressure measurement mechanism comprises a pressure transmitting lumen (or “pressure lumen”). Various devices within the telescoping assembly 1000 define one or more lumens that run the length of the devices that may be used to measure pressure at a distal end of each device.

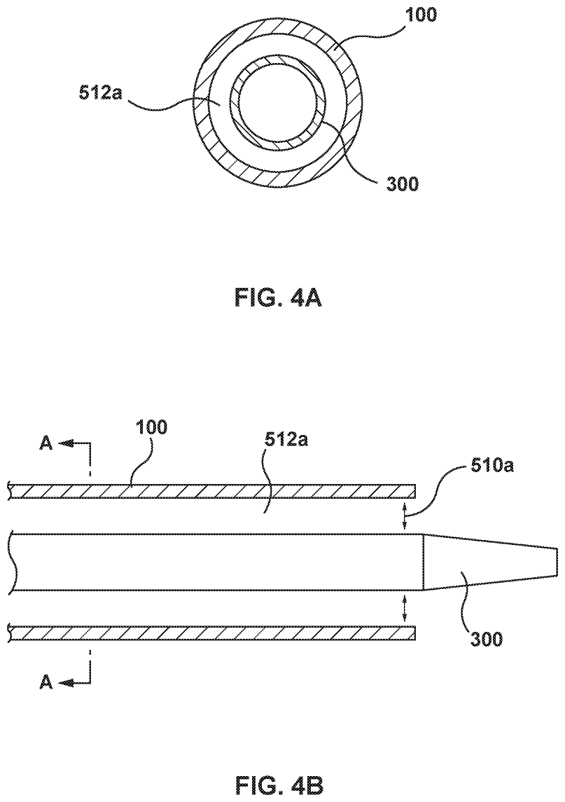

Referring to FIGS. 4A and 4B, in on embodiment, the telescoping assembly 1000 defines gaps between the various devices, i.e. a space exists between the outer diameters of an inner device and the inner diameter of an outer device. In one specific example, an outer diameter of the catheter 300, OD_{C}, is between 1.96 mm and 2.16 mm and the inner diameter of the sheath 100 ID_{S} is 3.23 mm and 3.43 mm which creates a gap 510a. In some embodiments, gap 510a runs the length of the catheter 300 and defines a pressure lumen 512a. FIG. 4A shows a cross section taken alone line A-A of FIG. 4B.

In some embodiments, the inner device is removed and there is no gap, and pressure lumen 512a is equal to the primary lumen of the device, in other words inner diameter, described below.

During a TIPS procedure, pressure lumen 512a fills with liquid, for example blood or saline. The distal opening of the pressure lumen 512a will be located in a first blood vessel, for example, hepatic vein 16, and the pressure of the liquid in the pressure lumen 512a will be substantially the same as the pressure in the first blood vessel.

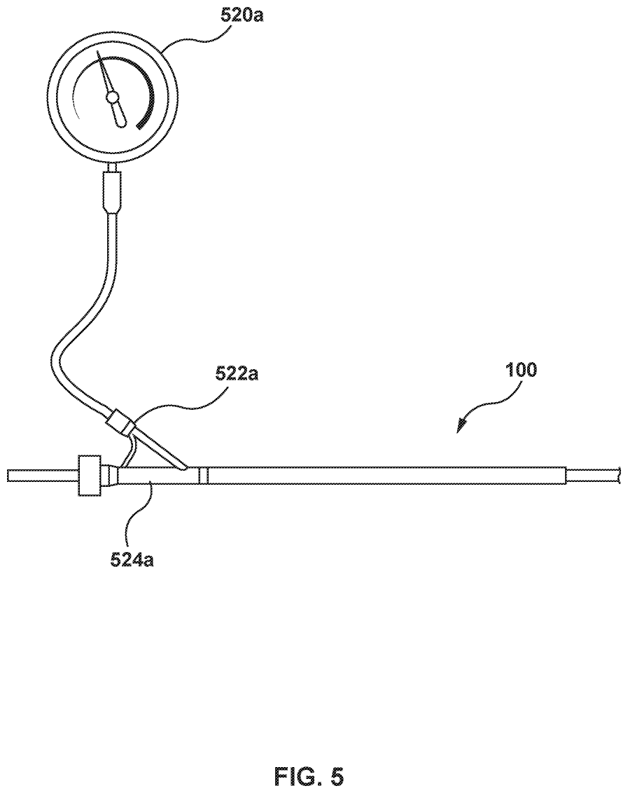

Referring to FIG. 5, in one embodiment, pressure transducer 520a is operable to be connected to the proximal end of one or more devices, for example, sheath 100, using connectors known by those skilled in the art, for example a luer-lock 522a and a Y-connection hub 524a. Pressure transducer 520a is operable to measure the pressure within the pressure lumen 512a. In one example, the distal end 126 of the sheath 100 is located within the hepatic vein 16 and the pressure in pressure lumen 512a is equal to the hepatic venous pressure.

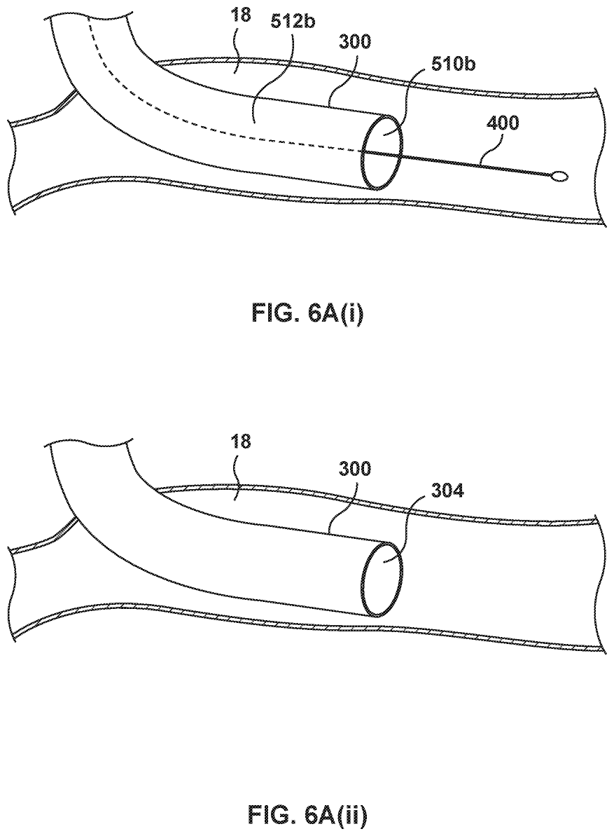

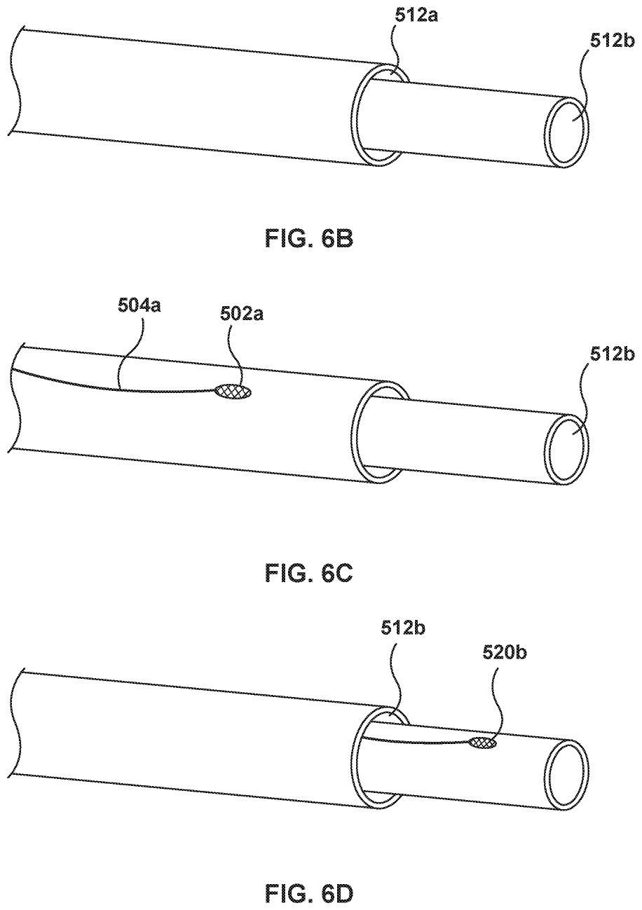

In such an embodiment, a second pressure lumen can be used to measure a second pressure in the portal vein 18. Referring to FIG. 6A(i), in one example, the inner diameter of the catheter 300 ID_{C} is greater than the outer diameter OD_{W} of the Puncture device 400, creating gap 510b and pressure lumen 512b. A second pressure transducer and a second fluid connector (not shown) can be connected to the proximal end of the catheter 300 in a similar manner as described above. In one such example, the distal end of the catheter 300 is located in the portal vein 18 and the pressure in pressure lumen 512b is equal to the portal pressure.

In another embodiment, puncture device 400 is removed from catheter 300, and the primary lumen 304 of catheter 300 is used as the pressure lumen 512b, as shown in FIG. 6A(ii).

As described above with respect to the pressure sensors 502, one or more pressure lumens 512 may be operatively coupled to a dedicated monitor 506 or other display device.

For clarity and completeness, some combinations of pressure sensors and pressure lumens that may be associated with the devices of telescoping assembly 1000 will now be described with reference to the figures.

As previously illustrated in FIG. 3A an embodiment of the telescoping assembly 1000 is shown where a single device comprises a pressure sensor 502 and has an associated pressure lumen 512, the pressure lumen being the main lumen or primary lumen of the device.

As previously illustrated in FIG. 3B an embodiment of the telescoping assembly 1000 is shown comprising an inner device and an outer device, wherein the inner device comprises a first pressor sensor 502a and the outer device comprises a second pressure sensor 502b.

As previously illustrated in FIG. 3C, an embodiment of the telescoping assembly 1000 is shown where a single device comprises two pressure sensors 502c and 502d.

As illustrated in FIG. 6B, an embodiment of the telescoping assembly 1000 is shown comprising an inner device and an outer device, wherein each device has an associated pressure lumen, 512a and 512b.

As illustrated in FIG. 6C, an embodiment of the telescoping assembly 1000 is shown comprising an inner device and an outer device, wherein the outer device comprises a pressure sensor 502a and inner device has an associated pressure lumen 512b.

As illustrated in FIG. 6D an embodiment of the telescoping assembly 1000 is shown comprising an inner device and an outer device, wherein the outer device has an associated pressure lumen 512a and the inner device comprises a pressure sensor 502b.

Other suitable combinations may be possible and the invention is not limited in this regard.

### Taper

When telescoped devices traverse through the relatively stiff liver tissue (crossing and dilating the tract), a gap between the devices may create a shoulder that may cause the devices to “snag” and fail to cross or be difficult to advance. There is also a risk of coring the tissue, i.e., pulling pieces of tissue off into the gap like a cookie cutter. In order to reduce these risks, the distal ends of one or more devices may be tapered. This allows for the presence of a lumen with a minimal or no gap at a distal end.

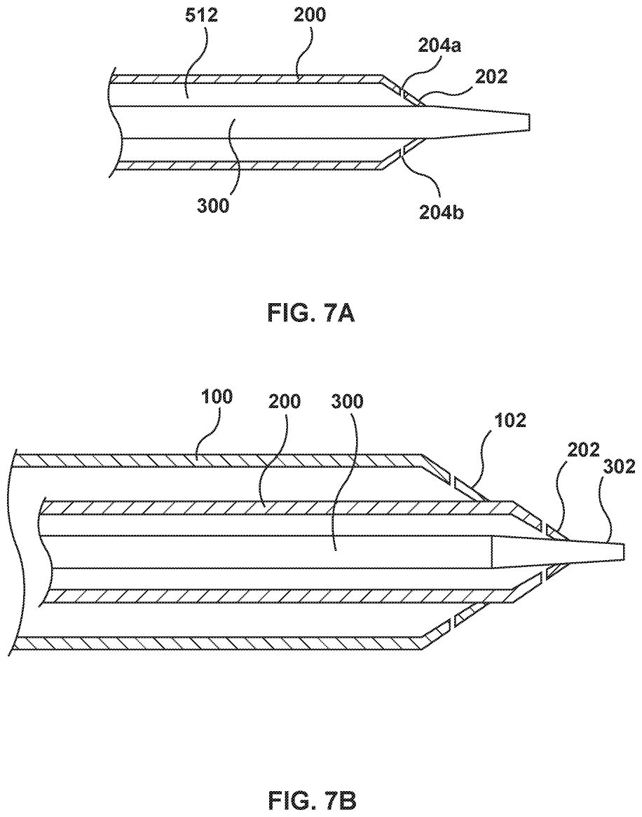

Referring now to FIG. 7A, in one embodiment of the present invention, the distal end of dilator 200 defines a tapered end 202. Tapered end 202 may comprise one or more holes or apertures 204, for example two apertures, 204a and 204b. In one specific embodiment, apertures 204 may be between 0.8 mm and 1.2 mm in diameter. The outer and inner diameters of tapered end 202 gradually decreases (in a distal direction, shown in FIG. 7A) until the dilator 200 inner diameter is substantially the same as the outer diameter of catheter 300. In other words, catheter 300 fills the gap or opening at the distal tip of tapered end 202 to create a smooth transition as the devices traverse through tissue. In other embodiments, one or more of the sheath 100 or catheter 300 may comprise a tapered end 102 and tapered end 302 respectively, as shown in FIG. 7B.

During a TIPS procedure, fluid may pass through apertures 204 to fill the device lumens with fluid, thereby creating pressure lumen 512. The pressure within the pressure lumen 512 may then be measured as described above, while mitigating the risks associated with advancement of the assembly through the liver.

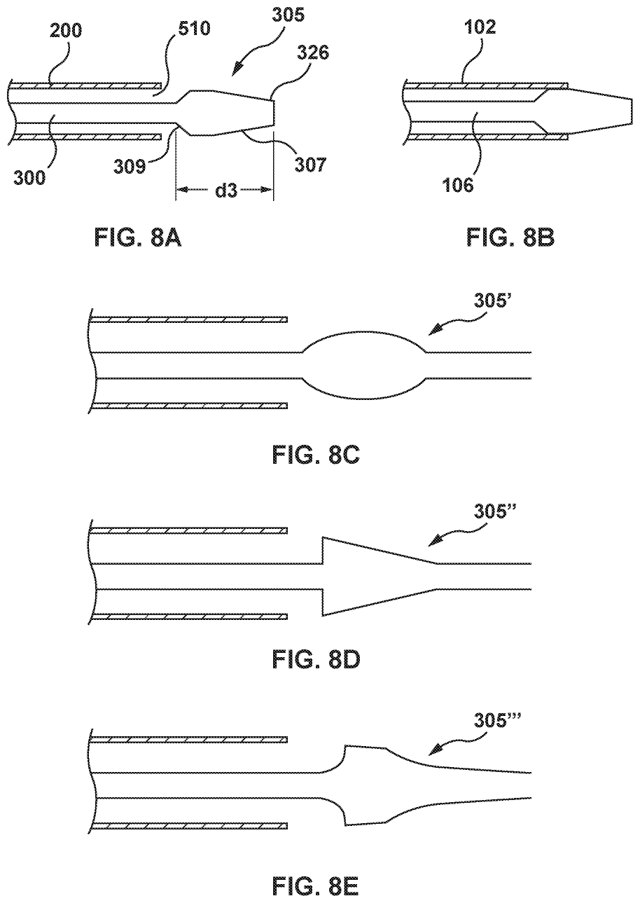

Referring now to FIGS. 8A and 8B, in another embodiment, one or more devices may comprise a varying-diameter portion defining a shape configured such that advancement and retraction of an inner device may selectively define a gap, with respect to the outer device, when the varying-diameter portion is in a first position and a tapered end when the varying-diameter portion is in a second position, respectfully.

In one example, catheter 300 comprises a varying-diameter portion 305 defining a distal taper 307 and a proximal taper 309 located proximally of the distal taper 307. In one embodiment, varying-diameter portion 305 is located substantially at the distal end 326 of catheter 300. As illustrated in FIG. 8A, catheter 300 has an outer diameter substantially along its length, and, at a distance D3 from the distal end 326, the outer diameter increases in the proximal taper 309. More specifically, the outer diameter increases in a distal direction until it reaches a portion where the outer diameter is substantially the same as inner diameter of the dilator 200.

As illustrated in FIG. 8B, when catheter 300 varying-diameter portion 305 is inserted within dilator 200 at a first location, there is substantially no gap between the distal end of proximal taper 309 and an inner wall of dilator 200. Further distally, at distal taper 307, the diameter reduces in a distal direction to create a tapered end towards the distal end 326 of the catheter 300.

In other embodiments, varying-diameter portion is located a longitudinal distance from distal end 326, for example, as shown in FIG. 8D. In other words, distal taper 307 does not begin at the distal end 326.

As described above, FIG. 8A illustrates a combination of a dilator 200 and catheter 300, with the catheter 300 shown in an advanced position. FIG. 8B illustrates the catheter 300 in a retracted position. In the retracted position, there is substantially no gap between the dilator 200 and catheter 300, and the two devices can be advanced through blood vessels or tissue while mitigating the risk of tissue coring. In the advanced position, gap 510 exists between the catheter 300 and dilator 200, and pressure lumen 512 can be used to measure a pressure in the dilator 200.

In use, the telescoping assembly 1000 is in the retracted position while it advances through the vasculature to a pre-determined position within a blood vessel. Subsequently, the catheter 300 is advanced distally within the dilator 200 to the advanced position, and a gap 510 and pressure lumen 512 are created. In other words, distal end of the dilator 200 remains substantially stationary while varying-diameter portion 305 of the catheter 300 advances distally. The pressure in pressure lumen 512 can then be measured in accordance with the techniques disclosed herein.

While FIGS. 8A and 8B show a dilator-catheter assembly, the varying-diameter portion 305 can be used in other combinations of the devices in the telescoping assembly 1000, for example sheath-dilator, sheath-catheter, catheter-wire, depending, for example, on the pressure being measured and which device is used to measure the pressure.

FIGS. 8C to 8E illustrate other embodiments of a varying-diameter portion 305. Alternative configurations for varying-diameter portion 305′, 305″, 305′″ are possible with an advanced position that defines a gap for pressure measurement, and a retracted position that defines a smooth transition for the telescoped devices to be advanced within the vessel.

### Single Lumen

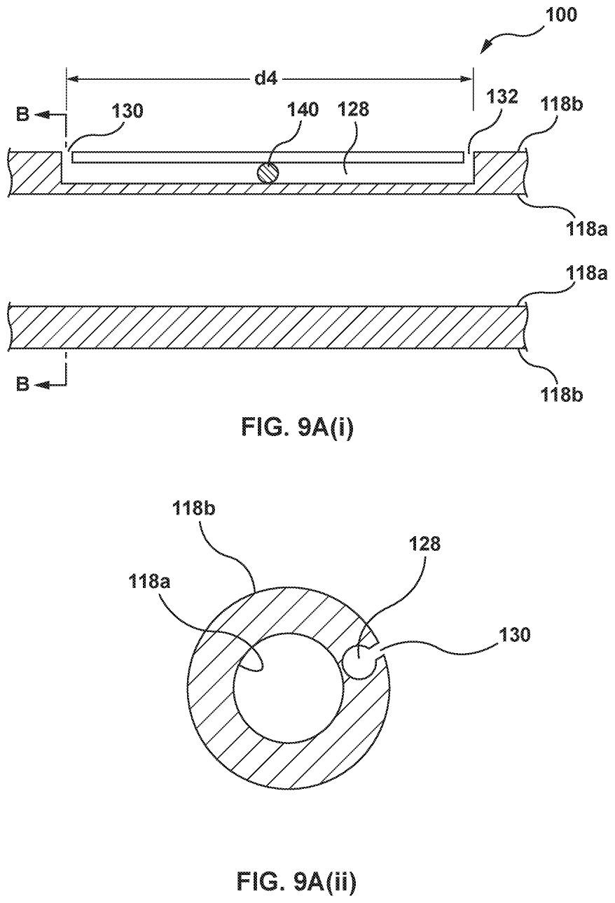

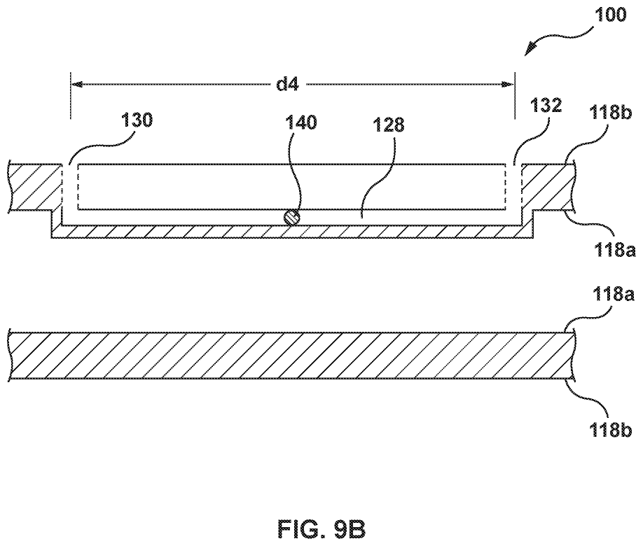

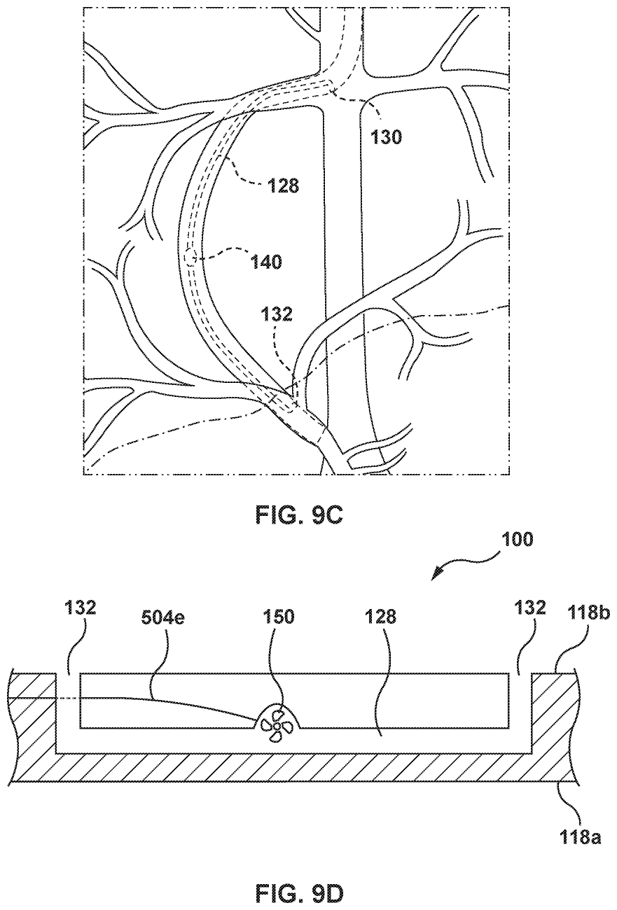

With reference now to FIGS. 9A(i) to 9B, devices and methods are disclosed for measuring a pressure differential using a single device in a procedure such as TIPS directly, i.e. reading a difference between the venous pressure and the portal pressure, as opposed to measuring the venous pressure, measuring the portal pressure, and then calculating the differential.

In one embodiment, sheath 100′ comprises an inner wall 118a and an outer wall 118b. Outer wall 118b defines a first opening 130 and a second opening 132. First opening 130 and second opening 132 are separated by a linear distance D4. FIG. 9A(ii) shows a cross section of the device of 9A(i) taken along line B-B.

Sheath 100′ further defines a pressure differential lumen 128, which is defined between the inner wall 118a and the outer wall 118b. Pressure differential lumen 128 extends between first opening 130 and second opening 132. In another embodiment, pressure differential lumen 128 is located substantially within inner wall 118a of sheath 100″, as shown in FIG. 9B.

In such embodiments, sheath 100′ comprises a pressure differential mechanism 140 located within pressure differential lumen 128. Pressure differential mechanism 140 is capable of measuring a differential between a first pressure measured at the first opening 130 and a second pressure measured at the second opening 132. In one example, pressure differential mechanism 140 is a differential pressure gauge 142.

In another embodiment, doppler imaging via an intravascular or transabdominal ultrasound probe may be used to measure flow velocities between the first pressure and the second pressure, which are correlated to a pressure differential.

The length D4 of the pressure differential lumen 128 is such that, in use, first opening 130 is positioned in a first blood vessel while second opening 132 is positioned in a second blood vessel. With reference to a TIPS procedure, as shown in FIG. 9C, in one example, first opening 130 is in the hepatic vein 16 and second opening 132 is the portal vein 18.

In another embodiment, pressure differential mechanism 140 comprises a turbine 150, as shown in FIG. 9D. Turbine 150 is configured and operable to rotate due to blood flow. The speed of the turbine correlates with the pressure differential, for example, the faster the blood is flowing, the faster the turbine is turning and the larger the pressure differential. The lower the differential, the slower the flow of blood and the slower the turbine would turn. In some embodiments, turbine 150 is connected to a wire 504e that extends the length of the device proximally to the handle, and which is then able to be electrically connected to monitor 506 or other device, which would output a measure of the turbine speed, the related blood flow velocity, and the estimated pressure differential. Monitor 506 may use electrical measurements of the turbine speed to calculate the PSG.

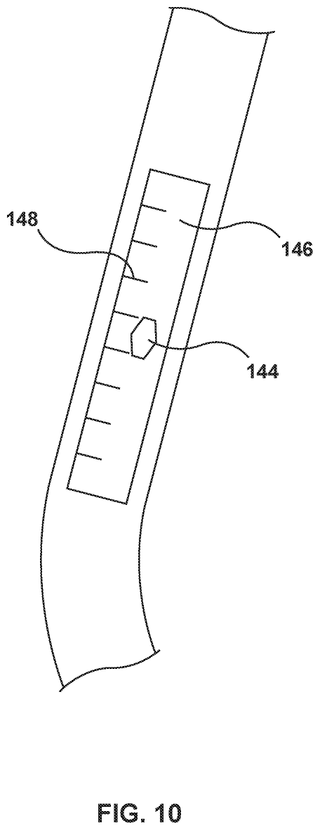

With reference to FIG. 10, in another embodiment, pressure differential mechanism 140 comprises a contrast bubble. In one embodiment, contrast bubble 144 may be an air (or other gas) bubble contained within a closed chamber 146, with chamber 146 being substantially filled with liquid such as a contrast solution visible using an imaging system, for example ultrasound or fluoroscopy. In another embodiment, contrast bubble 144 may be a balloon filled with contrast agent.

In a default state, that is when pressures through both first opening 130 and second opening 132 are substantially equal, the location of the contrast bubble is substantially centered in chamber 146, within a specified range. The location or position of the contrast bubble 144 may move distally or proximally as the pressure differential changes. For example, in use, as the pressure increases in the portal vein 18 relative to the hepatic vein 16, the bubble moves proximally along the device, i.e. toward the hepatic vein 16. As the pressure in the portal vein 18 decreases relative to the hepatic vein 16, the bubble moves distally along the device, i.e. toward the portal vein 18.

To assist a user in visualizing the position of contrast bubble 144, chamber 146 may have one or more markings 148 which are visible using an imaging system or modality for allowing a user to observe the position of the contrast bubble 144 as it moves due to pressure changes during the TIPS procedure. In one specific example, markings 148 may be radiopaque or made of some other material that will be visible on fluoroscopy.

In other embodiments, pressure differential lumen 128 and pressure differential mechanism 140 may be located on any of the telescoped devices that are large enough to accommodate them, for example, catheter 300 or dilator 200.

### Method of Measuring Pressures During a TIPS Procedure

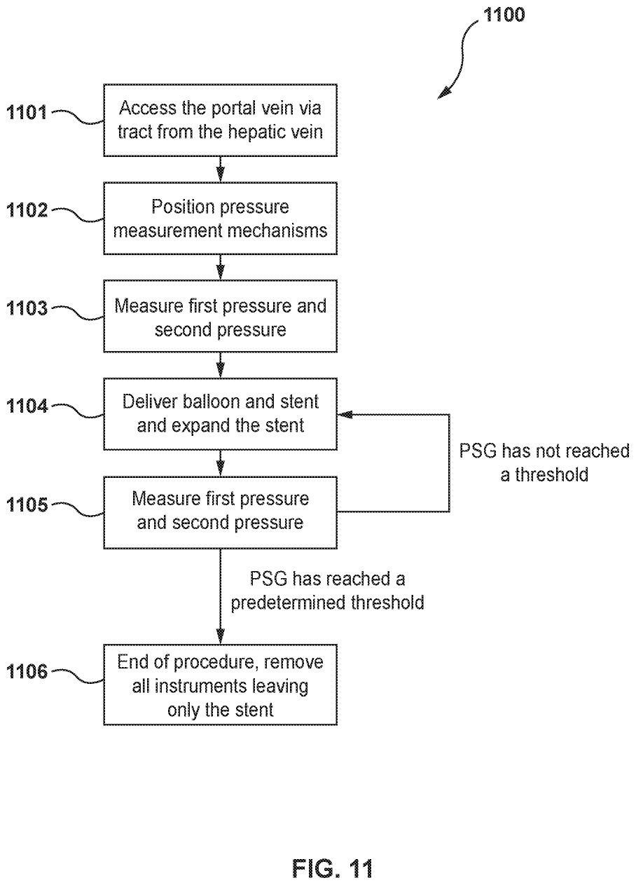

FIG. 11 shows a general method 1100 for measuring pressure at two locations within a patient's body. Specific reference is made to measuring pressures during a TIPS procedure.

The method begins at step 1101, after a tract has been created from the hepatic vein, through the liver, to the portal vein. In one example, the tract may have been created using an RF wire. In another example, the tract may have been created using a mechanical wire, needle, or stylet.

At step 1102, a first pressure measurement mechanism is positioned at a first location, for example the hepatic vein, and a second pressure measurement mechanism is positioned at a second location, for example in the portal vein. The pressure measurement mechanisms may be any of the type disclosed herein.

At step 1103, the hepatic venous pressure and portal pressures are measured in the hepatic vein and portal vein respectively. The two pressure measurements are taken using the first and second pressure measurement mechanisms, and the two measurements may be taken concurrently.

At step 1104, a balloon and stent are delivered to the tract. The stent is positioned and expanded to an initial diameter creating the portosystemic shunt. One or more devices comprising a pressure measurement mechanism may be removed prior to this step, depending on the device and pressure measurement mechanism used.

At step 1105, if necessary, a device and/or pressure measurement mechanism may be reintroduced if removed in the previous step, and the hepatic and portal pressures are measured a second time using the first and second pressure measurement mechanisms. If the portosystemic gradient has reached a predetermined threshold, the procedure ends at step 1106.

If the portosystemic gradient has not reached a predetermined threshold, then the method returns to step 1104 so that the stent can be expanded further.

### Device-Specific Method of Measuring Pressure

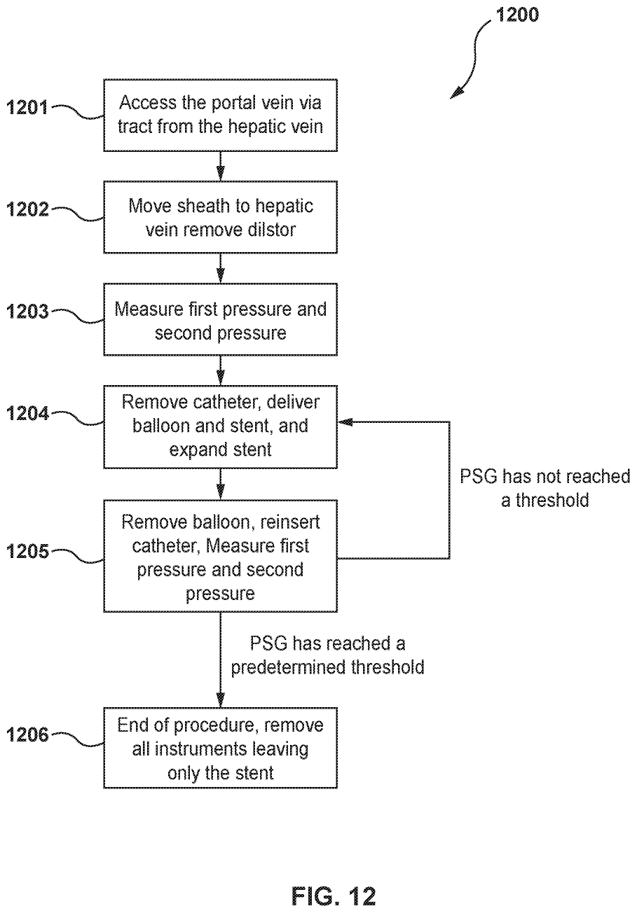

FIG. 12 shows method 1200 for measuring pressure, for example during a TIPS procedure, at two locations within a patient's body using a telescoping assembly 1000 comprising a sheath 100, dilator 200, catheter 300 and puncture device 400 (or guide wire).

At step 1201, the puncture device 400 is advanced into the portal vein, creating a tract through the liver. The position of puncture device 400 is confirmed, for example by known techniques such as visualizing the puncture device 400 using a medical imaging methodology such as ultrasound or by aspirating using a catheter 300. Puncture device 400 may be used as a guidewire for the TIPS procedure, or the puncture device 400 may be swapped for a guidewire at this step. (As an optional step, the dilator 200 and sheath 100 may be advanced to the portal vein over puncture device 400 to widen the tract through the liver)

At step 1202, the dilator 200 is removed and the distal end of the sheath 100 is positioned in the hepatic vein. (Optionally, the dilator 200 may remain and the sheath 100 and dilator 200 distal ends are positioned in the hepatic vein.) The telescoping assembly 1000 may be configured so that the catheter 300 is backloadable, i.e., the dilator 200 can be removed without removing the catheter 300. Position distal end of catheter 300 in the portal vein 18.

At step 1203, pressure is measured in both the hepatic vein 16 and portal vein 18. The two measurements may occur substantially concurrently. If the dilator 200 is removed, the hepatic venous pressure may be measured using the pressure lumen created between the sheath 100 and the catheter 300. Optionally, if the dilator 200 remains, the hepatic venous pressure may be measured using the pressure lumen created between dilator 200 and the catheter 300. Portal pressure may be measured using the pressure lumen created between the catheter 300 and the puncture device 400. Alternatively, the puncture device 400 may be removed from the telescoping assembly 1000, leaving only the catheter 300 in the portal vein, and the primary lumen of catheter 300 may be used to measure the portal pressure.

At step 1204 the catheter 300 is removed from the assembly and a balloon and stent are delivered to the intrahepatic tract. The stent is positioned and expanded to an initial diameter creating a portosystemic shunt. If the puncture device 400/guidewire was removed in step 1203, it would typically be reintroduced before the catheter 300 is removed.

At step 1205, the balloon is removed and the catheter 300 is reintroduced such that the distal end is positioned in the portal vein 18. The hepatic vein 16 and the portal vein 18 pressures are then measured again using the technique described at step 1203. If the portosystemic gradient (PSG), i.e. the difference between the hepatic venous pressure and portal pressure, has reached a predetermined threshold (for example <12 mmHG), the procedure ends at step 1206 and all devices of the telescoping assembly 1000 are removed, leaving only the stent.

If the portosystemic gradient has not reached the predetermined threshold, then the method returns to step 1204 where the balloon is reinserted, and the stent is expanded further.

Method 1200 has been described using pressure lumens, however, one skilled in the art would understand that any other pressure measurement mechanisms disclosed herein may be used, for example pressure sensors on one or multiple devices, a pressure differential mechanism within a lumen, or any combination of pressure lumens or pressure sensors. If pressure sensors are used, the number of device exchanges may be reduced as described herein.

The steps of measuring the pressures and calculating the portosystemic gradient may be done manually or may be automatic, for example the pressure measurement mechanisms may be connected to a monitor which may generate an alert, such as a visible or audible indication, to the user that the PSG has reached the predetermined threshold.

For clarity, measuring a first pressure means measuring a pressure at a first location and measuring a second pressure means measuring a pressure at a second location. The first location may be within a first blood vessel and the second location may be within a second blood vessel, or the first and second locations may be within the same blood vessel. During a procedure, the “first pressure” and “second pressure” may be measured multiple times at the first and second locations, respectively. Regardless of how many times they are taken, pressure measurements at the first and second locations are referred to as the first and second pressure measurements respectively.

Thus, as described hereinabove, aspects of the present invention comprise assemblies, systems, devices and methods for use in TIPS/DIPS and similar procedures where multiple pressure measurements may be required at multiple locations. Various embodiments have been described that provide advantages in such procedures, for example by allowing a pressure differential measurement to be made or a plurality of pressure measurements to be taken in a plurality of locations substantially without requiring movement of repositioning of one or more devices.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A telescoping assembly comprising:
a first elongate member;
a second elongate member moveable relative to the first elongate member when said second elongate member is inserted into said first elongate member;
a first pressure measurement mechanism associated with one of the first elongate member or the second elongate member; and
a second pressure measurement mechanism associated with one of the first elongate member or the second elongate member.

2. The telescoping assembly claim 1, wherein the first pressure measurement mechanism comprises a first pressure sensor and the second pressure measurement mechanism comprises a second pressure sensor.

3. The telescoping assembly of claim 1, wherein the first pressure measurement mechanism comprises a pressure transmitting lumen, the pressure transmitting lumen being adapted for fluid communication with a pressure transducer, and the second pressure measurement mechanism comprises a pressure sensor.

4. The telescoping assembly of claim 1, wherein the first pressure measurement mechanism comprises a first pressure transmitting lumen, the first pressure transmitting lumen being adapted for fluid communication with a first pressure transducer, and the second pressure measurement mechanism comprises a second pressure transmitting lumen, the second pressure transmitting lumen being adapted for fluid communication with a second pressure transducer.

5. The telescoping assembly of claim 2, wherein the first elongate member is selected from the group consisting of: a sheath; a dilator; and a catheter, and the second elongate member is selected from the group consisting of: a dilator; a catheter; and a guidewire.

6. The telescoping assembly of claim 5, wherein the first pressure sensor and the second pressure sensor are both associated with the first elongate member.

7. The telescoping assembly of claim 5, wherein the first pressure sensor and the second pressure sensor are both associated with the second elongate member.

8. The telescoping assembly of claim 3, wherein the pressure transmitting lumen is associated with the first elongate member, and the first elongate member is selected from the group consisting of a sheath; a dilator; and a catheter, and the pressure sensor is associated with the second elongate member and the second elongate member is selected from the group consisting of a dilator; a catheter; and a guidewire.

9. The telescoping assembly of claim 3, wherein the pressure transmitting lumen and the pressure sensor are both associated with the first elongate member, and the first elongate member is selected from the group consisting of: a sheath; a dilator; and a catheter.

10. The telescoping assembly of claim 3, wherein the pressure transmitting lumen and the pressure sensor are both associated with the second elongate member, and the second elongate member is selected from the group consisting of a dilator; and a catheter.

11. The telescoping assembly of claim 3, wherein the pressure sensor is associated with the first elongate member, and the first elongate member is selected from the group consisting of a sheath; a dilator, and the pressure transmitting lumen is associated with the second elongate member and the second elongate member is selected from the group consisting of a dilator; and a catheter.

12. The telescoping assembly of claim 4, wherein the first pressure transmitting lumen is associated with the first elongate member, and the first elongate member is selected from the group consisting of a sheath and a dilator, and wherein the second pressure transmitting lumen is associated with the second elongate member, and the second elongate member is selected from the group consisting of a dilator; and a catheter.

13. The telescoping assembly of claim 2, wherein the second elongate member comprises a balloon and the first pressure sensor is operatively located on one of a distal or proximal side of the balloon and the second pressure sensor is operatively located on another of the distal or proximal side of the balloon.

14. The telescoping assembly of any one of claim 5, 7, or 8, wherein the guidewire comprises a radiofrequency wire.

15. The telescoping assembly of any one of claim 5, 7, or 8, wherein the guidewire comprises a mechanical wire.

16. The telescoping assembly of any one of claims 1 to 13, wherein the first elongate member defines a tapered end defining one or more apertures.

17. The telescoping assembly of claim 16, wherein the second elongate member is selected from the group consisting of: a dilator; and a catheter, and the second elongate member defines a tapered end defining one or more apertures.

18. The telescoping assembly of claim 1, wherein the second elongate member defines a varying-diameter portion.

19. The telescoping assembly of claim 18, wherein, in an advanced position of the telescoping assembly, the varying-diameter portion is spaced distally from a distal end of the first elongate member, and in a retracted position of the telescoping assembly, the varying-diameter portion substantially abuts the distal end of the first elongate member.

20. The telescoping assembly of claim 19, wherein a gap is defined between a distal end of the first elongate member and the varying-diameter portion in the advanced position.

21. A system for use in an intrahepatic portosystemic shunt procedure comprising:
the telescoping assembly of any one of claims 1 to 13; and
a monitor for displaying at least one of: a first pressure associated with the first pressure measurement mechanism; a second pressure associated with the second pressure measurement mechanism; or a pressure differential between the first pressure and the second pressure.

22. The system of claim 21, wherein the monitor is configured to generate an alert when the pressure differential has reached a predetermined threshold.

23. The system of claim 22, wherein the alert is an audible alert.

24. The system of claim 22, wherein the alert is visual alert.

25. A medical device comprising:
an elongate member comprising an inner wall and an outer wall;
a first opening defined by the outer wall;
a second opening defined by the outer wall, spaced apart longitudinally from the first opening;
a lumen, defined between the inner wall and the outer wall, the lumen extending between the first opening and the second opening; and
a pressure differential mechanism located within the lumen for measuring a pressure differential between a first pressure at the first opening and a second pressure at the second opening.

26. The medical device of claim 25, wherein the pressure differential mechanism comprises a pressure sensor.

27. The medical device of claim 25, wherein the pressure differential mechanism comprises a contrast bubble.

28. The medical device of claim 25, wherein the pressure differential mechanism comprises a turbine.

29. The medical device of claim 25, wherein the pressure differential mechanism comprises a balloon.

30. The medical device of claim 25, wherein the elongate member is selected from a group consisting of a sheath, a dilator, and a catheter.

31. A system for use in an intrahepatic portosystemic shunt procedure comprising:
the medical device of any of claims 25 to 30; and
a monitor for displaying at least one of: the first pressure associated with the first opening; the second pressure associated with the second opening; and a pressure differential between the first pressure and the second pressure.

32. The system of claim 31, wherein the monitor is configured to generate an alert when the pressure differential has reached a predetermined threshold.

33. The system of claim 32, wherein the alert is an audible alert.

34. The system of claim 32, wherein the alert is visual alert.

35. A method of measuring pressure during an intrahepatic portosystemic shunt procedure, the method comprising:
measuring a first pressure at a first location associated with the intrahepatic portosystemic shunt procedure within a patient's body using a first pressure measurement mechanism; and
measuring a second pressure at a second location associated with the intrahepatic portosystemic shunt procedure within the patient's body using a second pressure measurement mechanism;
wherein the first pressure and the second pressure are measured while substantially maintaining a position of the first pressure measurement mechanism at the first location and maintaining a position of the second pressure measurement mechanism at the second location.

36. The method of claim 35 wherein the steps of measuring the first pressure and measuring the second pressure are performed substantially concurrently.

37. The method of claim 35 wherein the first pressure is a venous pressure and the second pressure is a portal pressure.

38. The method of claim 35, wherein the first location is one selected from the group of: a hepatic vein, a right atrium, and an inferior vena cava.

39. The method of claim 35, wherein the second location is a portal vein.

40. The method of claim 35, further comprises a step of positioning the first pressure measurement mechanism at the first location and positioning the second pressure measurement mechanism at the second location.

41. The method of claim 35 further comprising a step of creating a channel through tissue in order to access the second location.

42. The method of claim 41, wherein the channel is created using radiofrequency energy.

43. The method of claim 41, wherein the channel is created using mechanical energy.

44. The method of claim 35 further comprising a step of generating an alert when a pressure differential between the first pressure and the second pressure has reached a predetermined threshold.

45. A method of measuring a pressure differential during an intrahepatic portosystemic shunt procedure, the method comprising:
measuring a pressure differential between a first pressure at a first location and a second pressure at a second location, the first and second locations being associated with the intrahepatic portosystemic shunt procedure within a patient's body, using a pressure differential mechanism.
